(19) United States
(12) Patent Application Publication
(10) Pub. No.: US 2021/0038426 A1
(43) Pub. Date: Feb. 11, 2021

(54) DEVICE AND METHOD FOR CONTROLLING THE MOVEMENT OF AN OCULAR THERAPY APPARATUS INCLUDING AN ARTICULATED SUPPORT ARM

(71) Applicant: KERANOVA, SAINT ETIENNE (FR)

(72) Inventors: Nicolas BOULAROT, SAINT GENEST LERPT (FR); Fabrizio ROMANO, BEYNOST (FR)

(21) Appl. No.: 16/964,229

(22) PCT Filed: Jan. 25, 2019
(86) PCT No.: PCT/EP2019/051876

§ 371 Date: Jul. 23, 2020

(30) Foreign Application Priority Data
Jan. 25, 2018 (FR) 1850578

(51) Int. Cl. A61F 9/008; A61B 34/32

(52) U.S. Cl. CPC A61F 9/008; A61F 2009/00846; A61F 2009/00844; A61B 34/32

(57) ABSTRACT

The present invention relates to a device for controlling the movement of an ocular therapy apparatus of the type comprising an articulated support arm (2), of which the free end is intended to be placed opposite an ocular tissue, an acquisition system (4) mounted on the arm (2) for acquisition of a measurement pair comprising an image of the ocular tissue, and a signal representative of a vertical distance along the axis Z between the end of the arm and the ocular tissue, characterized in that the control device (5) comprises means for actuating the acquisition system (4), means for processing each measurement pair acquired, and servo means for moving the free end of the arm (2) between an initial position and a desired final position.

### TECHNICAL FIELD

The present invention relates to the general technical field of the treatment of ocular pathologies by using therapy equipment intended to perform operations on the eye, and more particularly:
operations on the anterior segment of the eye such as the cataract (at the level of the crystalline lens), and/or
refractive surgery operations (at the level of the cornea), and/or
operations intended to treat glaucoma or other retinal pathologies.

More specifically, the invention relates to a device and a method for monitoring the movement of an ocular pathology treatment system mounted on an articulated robotic arm to allow its movement along three orthogonal axes X, Y and Z.

In general, the present invention finds an application, when a therapy equipment will act in the eye on the surface or in depth by means of physical agents (such as light waves, ultrasounds, microwaves, etc.), whose path must be accurately controlled, in order to reach the target without damaging the adjacent structures.

In the following, the invention will be described with reference to therapy equipment including an articulated robotic arm integrating a system for cutting a human or animal tissue, such as a cornea, or a crystalline lens, by means of a femtosecond laser.

It is however very obvious to those skilled in the art that the invention described below can be used for the monitoring of the movement of an articulated robotic arm integrating any other type of system for treating an ocular pathology.

### PRIOR ART

There are many therapy equipment items including a laser for the treatment of ocular pathology. The laser is then used as an optical scalpel.

Such a laser is capable of making incisions on the transparent tissues of the eye, in depth, without using surgical instruments. It has the advantage of being quick and well tolerated, but above all of eliminating the manual surgical procedure which is operator-dependent.

Thus, the surgery performed with a laser becomes extremely accurate and repeatable. It provides a guarantee of safety which cannot be achieved with a gesture performed by a human operator, so that the use of a laser allows considering a quasi-automated surgery, where the machine will carry out steps of the surgical procedure instead of the practitioner.

In order for therapy equipment including a laser to carry out steps of a treatment procedure, two essential phases must be implemented beforehand:
i) Attaching the therapy equipment to the eye, in order to prevent the eye movements during the treatment procedure, and in order to align the axis of the eye with the reference frame of the machine;
   in this way, the machine and the eye are aligned and secured to each other, and the treatment can start safely, without danger of deflection or movement during the procedure,
ii) Performing a mapping of the intraocular structures of the patient's eye by means of an integrated imaging system such of the OCT (optical coherence tomography) or Scheimpflug (visible light mapping), or UBM (Ultrasonic Bio Microscopy) type in order to contour the areas that will be reached by the laser beam so that they can be cut or fragmented.

To carry out step i), it is necessary to position on the patient's eye an immobilization member equipped with a suction ring capable of suctioning the eye and holding it firmly in position.

At present, the therapy equipment acting in the eye and requiring immobilization of the eyeball during the phases i) and ii) (then during the treatment phase) are all equipped with an immobilization member manipulated manually by the operator.

Such therapy equipment has many disadvantages:
the manual positioning of the immobilization member is subject to some variability, which depends on many factors; the quality of the positioning of the immobilization member varies in particular from one operator to another;
   this induces variability in the conditions of immobilization of the patients, knowing that the quality of the treatment is very dependent on the quality of the positioning of the immobilization member,
the time required for the operator to position an immobilization member (such as a surgeon) is highly valued and therefore very expensive, for a gesture that could be assigned to a machine, which would do it in a more accurate manner, in a repeatable way and at a much lower cost,
the manipulation of the immobilization member is often difficult to perform, since the operator is not placed in optimal conditions and is often hampered by different obstacles to observe the eyeball and know whether the positioning of the immobilization member is correct or not,
the ability of the operator to judge the proper positioning of the ocular immobilization member, view which must be based on indications using benchmarks in space (centering level, presence of a tilt, of a rotation, etc.) is much lower than that of a machine which is equipped with sensors and imaging systems capable of correcting the X, Y or Z or the trim positioning faults, with extremely accurate levels of definition,
the patients may, depending on the delicacy of the operator, experience discomfort, injury, or improper positioning of the immobilization member likely to compromise the effectiveness of the treatment.

An aim of the present invention is to propose an intelligent and autonomous system, having robotic movements, vision, sensors and abilities to interpret the images generated by the integrated vision, to automate the phase of positioning the eyeball immobilization member.

### DISCLOSURE OF THE INVENTION

To this end, the invention relates to a device for monitoring the movement of an ocular therapy apparatus of the type comprising:
a support arm, the free end of the arm being intended to come in line with a human or an animal ocular tissue, said arm being articulated to allow the movement of the free end of the arm along three orthogonal axes X, Y and Z two by two:
   the axis X, defining a horizontal, longitudinal direction,
   the axis Y, defining a horizontal, transverse direction, which with the axis X defines a horizontal plane XY,
   the axis Z, defining a vertical direction, perpendicular to the horizontal plane XY,
an acquisition system mounted on the arm for the acquisition of a measurement pair including:
   an image of the ocular tissue, and
   a signal representative of a vertical distance along the axis Z between the end of the arm and the ocular tissue, remarkable in that the monitoring device comprises:
means for controlling the acquisition system for the acquisition of a plurality of measurement pairs successively over time,
means for processing each measurement pair, said processing means including:
   means for estimating, from the current measurement pair, the vertical distance along the axis Z between the end of the arm and the ocular tissue,
   means for calculating, from the image of the current measurement pair, a horizontal deviation between:
      a current horizontal position of the free end of the arm in the horizontal plane XY, and
      a desired final horizontal position of the free end of the arm in the horizontal plane XY,
servo-control means for:
   generating, if the calculated horizontal deviation is greater than a first threshold value, an instruction to horizontally move the arm in the horizontal plane XY in order to reduce the deviation between the current horizontal position and the desired final horizontal position,
   generating, if the calculated horizontal deviation is less than the first threshold value and if the estimated vertical distance is greater than a second threshold value, an instruction to vertically move the arm along a vertical direction in order to reduce the distance between the free end of the arm and the ocular tissue,
   generating, if the calculated horizontal deviation is less than the first threshold value and if the measured vertical distance is less than the second threshold value, an instruction to immobilize the arm.

Thus, the invention allows making the positioning phase of the therapy equipment more accurate, repeatable and at a lower cost than the existing solutions.

Preferred but non-limiting aspects of the monitoring device are the following:
the calculation means may comprise:
   means for detecting, from the image of the current measurement pair, the horizontal position of at least one point of interest of the ocular tissue,
   means for evaluating, from the detected horizontal position of the point of interest, a horizontal deviation between:
      the current horizontal position of the free end of the arm in the horizontal plane XY, and
      the desired final horizontal position of the free end of the arm in the horizontal plane XY;
the detection means can be able to identify the ocular tissue in the acquired image, by the implementation of a shape recognition algorithm in order to detect three concentric circles in the image;
the therapy apparatus may further comprise a force sensor mounted on the free end of the arm to measure a mechanical force applied to the free end of the arm:
   the processing means comprising means for comparing said measured mechanical force with a third threshold value to determine whether the free end of the arm is in contact with an element that obstructs a vertical movement of the arm along the axis Z,
   the servo-control means being programmed for generating an instruction to immobilize the arm if the measured mechanical force is greater than the third threshold value;
the acquisition system may comprise, for the acquisition of a signal representative of a vertical distance along the axis Z:
   means for acquisition by laser ranging, and/or
   means for acquisition by ultrasounds
   means for acquisition by image processing;
the servo-control means can be programmed to generate elementary movement instructions to allow the movement of the arm between its current position and a desired final position, said servo-control means generating an immobilization instruction subsequent to each elementary movement instruction.

The invention also relates to a method for monitoring the movement of an ocular therapy apparatus of the type comprising:
a support arm, the free end of the arm being intended to come in line with a human or an animal ocular tissue, said arm being articulated to allow the movement of the free end of the arm along three orthogonal axes X, Y and Z two by two:
   the axis X, defining a horizontal, longitudinal direction,
   the axis Y, defining a horizontal, transverse direction, which with the axis X defines a horizontal plane XY,
   the axis Z, defining a vertical direction, perpendicular to the horizontal plane XY;
an acquisition system mounted on the arm for the acquisition of a measurement pair including:
   an image of the ocular tissue, and
   a signal representative of a vertical distance along the axis Z between the end of the arm and the ocular tissue, remarkable in that the monitoring method comprises the following phases:
acquiring a plurality of measurement pairs successively over time via the acquisition system,
processing each measurement pair, the processing phase comprising the steps consisting of:
   estimating, from the current measurement pair, the vertical distance along the axis Z between the end of the arm and the ocular tissue,
   calculating, from the image of the current measurement pair, a horizontal deviation between:
      a current horizontal position of the free end of the arm in the horizontal plane XY, and
      a desired final horizontal position of the free end of the arm in the horizontal plane XY,
servo-controlling the movement of the arm by:
   generating, if the calculated horizontal deviation is greater than a first threshold value, an instruction to horizontally move the arm in the horizontal plane XY in order to reduce the deviation between the current horizontal position and the desired final horizontal position,
   generating, if the calculated horizontal deviation is less than the first threshold value and if the estimated vertical distance is greater than a second threshold value, an instruction to vertically move the arm along a vertical direction in order to reduce the distance between the free end of the arm and the ocular tissue,
   generating, if the calculated horizontal deviation is less than the first threshold value and if the measured vertical distance is less than the second threshold value, an instruction to immobilize the arm.

Preferred but non-limiting aspects of the monitoring method are the following:
the calculation step may include the following sub-steps:
   detecting, from the image of the current measurement pair, the horizontal position of at least one point of interest of the ocular tissue,
   evaluating, from the detected horizontal position of the point of interest, a horizontal deviation between:
      the current horizontal position of the free end of the arm in the horizontal plane XY, and
      the desired final horizontal position of the free end of the arm in the horizontal plane XY;
the detection sub-step can consist in identifying the ocular tissue in the acquired image, by the implementation of a shape recognition algorithm to detect three concentric circles in the image;
the therapy apparatus may further comprise a force sensor mounted on the free end of the arm for measuring a mechanical force applied to the free end of the arm:
   the processing phase comprising a step of comparing said measured mechanical force with a third threshold value to determine whether the free end of the arm is in contact with an element that obstructs a vertical movement of the arm along the axis Z,
   the servo-control step including the generation of an instruction to immobilize the arm if the measured mechanical force is greater than the third threshold value;
the acquisition phase can comprise:
   the acquisition, by laser ranging, of a signal representative of a vertical distance along the axis Z, and/or
   the acquisition, by ultrasounds, of a signal representative of a vertical distance along the axis Z, and/or
   the extraction of an acquired image from a signal representative of a vertical distance along the axis Z;
the servo-control step can include:
   generating an elementary movement instruction to allow the movement of the arm between its current position and a desired final position,
   generating an immobilization instruction subsequent to each elementary movement instruction,
   repeating the previous sub-steps until the calculated horizontal deviation is less than the first threshold value and the measured vertical distance is less than the second threshold value.

### BRIEF DESCRIPTION OF THE DRAWINGS

Other characteristics and advantages of the invention will emerge clearly from the following description of several alternative embodiments, given by way of non-limiting examples, from the appended drawings wherein:

FIGS. 1 and 2 illustrate a therapy apparatus including a support arm and a monitoring device according to the invention, the arm being:
in a retracted position in FIG. 1, and
In a deployed position in FIG. 2,

FIG. 3 schematically illustrates a cutting system integrated into the therapy apparatus,

FIG. 4 schematically illustrates the steps of a monitoring method implemented in the monitoring device,

FIG. 5 schematically illustrates the steps of moving the arm during a procedure for treating an ocular pathology.

### DETAILED DISCLOSURE OF THE INVENTION

The invention relates to a device and method for monitoring the movement of a therapy apparatus for a human or an animal ocular tissue. In the following description, the invention will be described, by way of example, for the cutting of an ocular tissue, it being understood that the present invention can be used for any other type of ocular treatment.

Referring to FIG. 1, an example of a therapy apparatus is illustrated.

The therapy apparatus comprises:
a movable box 1,
an articulated support arm 2 mounted on the box 1,
a cutting system mounted on the arm 2,
a force sensor 3 mounted at a free end of the arm 2,
an acquisition system 4 mounted on the arm 2 for the acquisition of images and signals representative of a distance between the free end of the arm 2 and the ocular tissue,
a monitoring device 5 integrated into the box 1, the monitoring device 5 including control means and processing means.

### 1. MOVABLE BOX

The box 1 allows the movement of the therapy equipment. It comprises in particular wheels 11, a metal frame and an appropriate fairing so as to present a minimum of recesses in order to prevent dust or pathogenic elements from lodging therein and developing.

The box 1 preferably comprises means for immobilization with respect to the ground to prevent its movement during surgical intervention.

The box 1 carries the various elements of the therapy equipment—such as the arm 2 and the monitoring device 5—and comprises means for their supply with electrical energy.

The box 1 can further comprise display and input means 12—such as a planning console—allowing the practitioner to control the therapy equipment and/or follow the progress of the treatment applied to the patient's eye.

Finally, the box 1 can include communication means 13 with or without wire for the exchange of data with a remote workstation (not represented), or with the monitoring device 5 if the latter is not integrated into the box 1.

### 2. SUPPORT ARM

The arm 2 comprises several arm segments 21-24 connected by articulations 25-27 (pivot or ball-joint connections) to allow the movement in rotation of the different segments 21-24 relative to each other.

Each articulation 25-27 includes a motorization and a brake. Advantageously, each brake is of the active type in the case of absence of an electrical energy supply. This allows preventing any unexpected movement of the arm, for example in the event of a system failure or power outage.

The motorizations and brakes of the articulations of the arm allow:
an automatic movement of the arm segments 21-24 relative to the box 1, and
the immobilization of the arm segments 21-24 relative to the box 1.

Particularly, the arm is articulated to allow the movement of the free end of the arm along three orthogonal axes X, Y and Z:
the axis X, defining a horizontal longitudinal direction,
the axis Y, defining a horizontal transverse direction, which with the axis X defines a horizontal plane XY,
the axis Z, defining a vertical direction, perpendicular to the horizontal plane XY.

The free end of the arm 2 includes an immobilization member equipped with a suction ring capable of suctioning the ocular tissue and holding it firmly in position. The monitoring device and method described below allow automatically positioning the immobilization member on the ocular tissue to be treated.

As illustrated in FIGS. 1 and 2, the arm 2 is able to move between:
a retracted position (FIG. 1) facilitating its transportation from one intervention room to another and/or inside an intervention room, and
an initial deployed position (FIG. 2) prior to the positioning of its free end on the ocular tissue to be treated.

The arm 2 is for example a TX260L marketed by the company STAUBLI.

The movement of the arm 2 is monitored by the monitoring device 5 which:
determines at all times the current position in the space of the free end of the arm,
generates movement instructions in order to adjust the current position of its free end by activating one or more motor(s) to reach a desired final position—position in which the immobilization member is centered and in contact with the ocular tissue,
generates instructions to immobilize the arm in order to keep the stationary arm by activating the brakes.

Advantageously, the arm may comprise declutching means to allow its movement manually, for example in the event of a failure or a power outage.

### 3. CUTTING SYSTEM

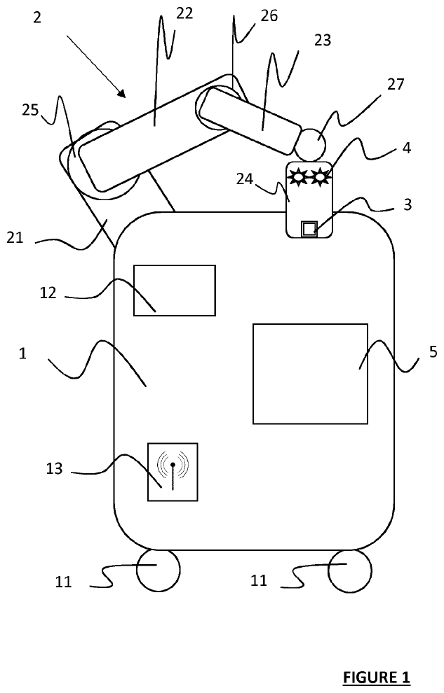

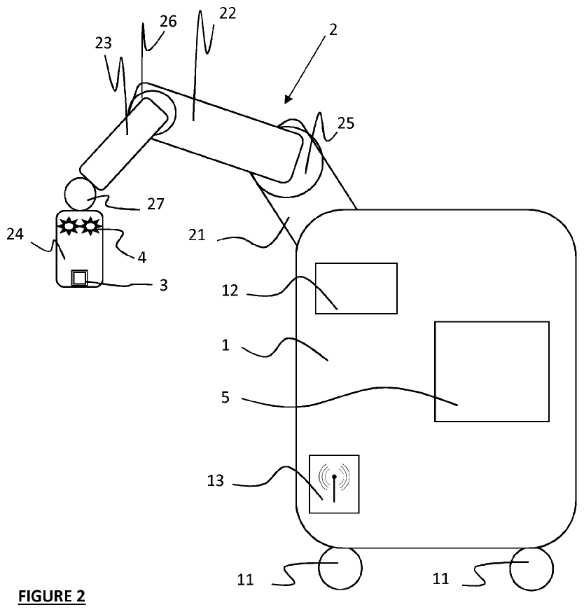

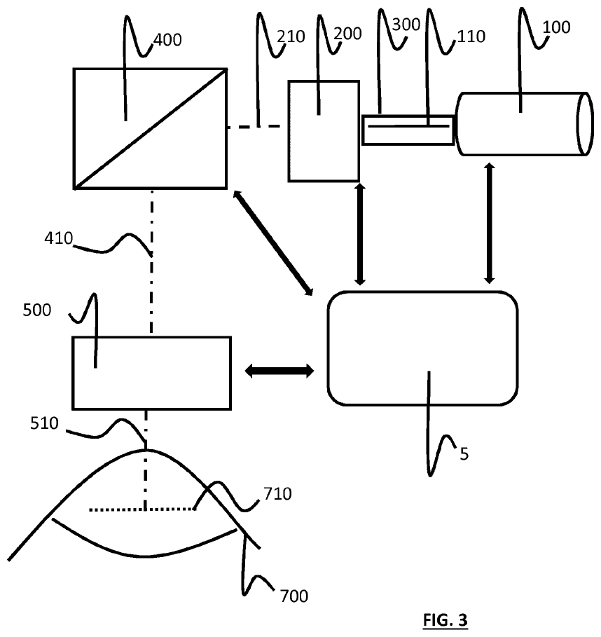

Referring to FIG. 3, there is illustrated one embodiment of a cutting system usable with the therapy apparatus according to the invention. The cutting system comprises:
a femtosecond laser 100,
a shaping system 200—such as a liquid-crystal Spatial Light Modulator (or SLM)—positioned downstream of the femtosecond laser 100,
an optical coupler 300 between the femtosecond laser 100 and the shaping system 200,
an optical scanner 400 downstream of the shaping system 200,
an optical focusing system 500 downstream of the optical scanner 400.

The monitoring device 5 allows piloting the shaping system 200, the optical scanner 400 and the optical focusing system 500.

The femtosecond laser 100 is able to emit an initial LASER beam in the form of pulses. By “femtosecond laser” is meant a light source able to emit a LASER beam in the form of ultra-short pulses, the duration of which is comprised between 1 femtosecond and 100 picoseconds, preferably between 1 and 1000 femtoseconds, in particular on the order of around a hundred femtoseconds.

The shaping system 200 extends over the path of the initial LASER beam 110 derived from the femtosecond laser 100. It allows transforming the initial LASER beam 110 into a modulated LASER beam 210. More specifically, the shaping system allows modulating the phase of the LASER beam 110 to distribute the energy of the LASER beam into a plurality of impact points in its focal plane, this plurality of impact points defining a pattern. In other words, the shaping system 200 allows modulating the final energy distribution of the LASER beam in the focusing plane 710 corresponding to the tissue 700 cutting plane. It is adapted to modify the spatial profile of the wave front of the primary LASER beam 110 derived from the femtosecond laser 100 in order to distribute the energy of the LASER beam at different focal points in the focusing plane 710. The shaping system 200 therefore allows, from a Gaussian LASER beam generating a single impact point, and by means of the phase mask, distributing its energy by phase-modulation so as to simultaneously generate several impact points in its focusing plane from a single LASER beam shaped though phase-modulation (a single beam upstream and downstream of the SLM).

The optical coupler 300 allows transmitting the LASER 110 beam derived from the femtosecond laser 100 towards the shaping system 200. It advantageously comprises an optical fiber, in particular a hollow-core Photonic-Crystal Fiber (PCF). A hollow-core photonic crystal fiber is an optical fiber which guides light essentially inside a hollow region (the core of the fiber), so that only a minor part of the optical power propagates in the solid fiber material (typically a glass). The appeal for the hollow-core photonic crystal fibers are mainly that the primary guidance in the hollow region minimizes the non-linear effects of the modulated LASER beam and allows a high damage threshold. Advantageously, the hollow region of the hollow-core photonic crystal fiber can be placed under vacuum to limit the propagation losses of the LASER beam derived from the femtosecond laser 100. To this end, the optical coupler 300 comprises first and second connection cells sealingly mounted at each end of the hollow-core photonic crystal fiber. These connection cells are connected to a vacuum pump P integrated into the casing 1 to put the hollow core of the optical fiber under vacuum by pumping at the connection cells. The fact of carrying out a vacuum pumping at each end of the optical fiber 31 allows facilitating the vacuuming of the hollow core over the entire length of the optical fiber 31.

The optical scanner 400 allows orienting the modulated LASER beam 210 to move the pattern along a movement path predefined by the user in a focusing plane 710.

The optical focusing system 500 allows moving the focusing plane 710—corresponding to the cutting plane—of the deflected LASER beam 410 derived from the optical scanner 400.

Advantageously, the shaping system 200, the optical scanner 400 and the optical focusing system 500 can be mounted in a compartment fixed to the end 24 of the arm, while the femtosecond laser can be integrated into the box 1, the optical coupler 300 extending between the box 1 and the end segment 24 to propagate the initial laser beam 110 between the femtosecond laser 100 and the shaping system 200.

### 4. FORCE SENSOR

The force sensor 3 allows detecting mechanical forces generated in opposition to a movement of the arm 2, these forces which reflect the presence of an obstacle and which may correspond to obtaining a contact between the end of the arm 2 and the ocular tissue. The force sensor 3 can be mounted on the end segment 24 of the arm 2.

The force sensor 3 is of a type known per se to those skilled in the art. It is able to capture and measure compressive and tensile forces applied along the longitudinal axis of the end segment 24 of the arm 2. It comprises one (or more) strain gauge(s) mounted on the end segment 24 of the arm 2.

Each mechanical force measured by the force sensor 3 is transmitted to the monitoring device 5.

When the value of the measured mechanical force is greater than a threshold value, the monitoring device performs one or more predetermined action(s) (generation of an instruction to immobilize the arm, order of emission of a visual stimulus on display and input means 12, and/or of an auditory stimulus on a loudspeaker integrated into the box, etc.).

### 5. ACQUISITION SYSTEM

The acquisition system 4 allows acquiring measurement pairs used to monitor the movement of the arm 2 relative to the ocular tissue to be treated.

Each measurement pair comprises one (or more) image(s) of an area located facing the free end of the arm 2.

To this end, the acquisition system 4 may comprise an image acquisition unit of the OCT (Optical Coherence Tomography) or Scheimpflug (visible light mapping), or UBM (Ultrasonic Bio Microscopy) type. Such an image acquisition unit can be mounted on the end segment 24 of the arm 2, for example upstream of the optical scanner 400. This image acquisition unit is arranged so as to have a sufficiently wide acquisition field (for example observe a perimeter P corresponding to a square of a side of 50 cm at a distance of 30 cm) in order to be able to identify the ocular tissue in this acquisition field. Advantageously, the image acquisition unit can be equipped with (coaxial or non-coaxial) lighting means in order to facilitate recognition of the ocular tissue.

Each measurement pair also comprises one (or more) signal(s) representative of a distance between the free end of the arm 2 and the ocular tissue.

To this end, the acquisition system 4 may comprise a laser ranging unit or an ultrasonic ranging unit or an image-analysis ranging unit or a ranging by any other equivalent device known to those skilled in the art capable of acquiring a signal representative of a distance between the free end of the arm 2 and the object located facing this end. Such a ranging unit can also be mounted on the end segment 24 of the arm 2.

### 6. MONITORING DEVICE

The monitoring device 5 allows:
processing the measurement pairs derived from the acquisition system as well as the forces measured by the force sensor 3, and
piloting the various elements constituting the therapy apparatus (arm 2, cutting system (in particular femtosecond laser 100, shaping system 200, scanner 400, optical focusing system 500, vacuum pump of the optical coupler 300, etc.), force sensor 3, acquisition system 4, etc.).

The monitoring device 5 is connected to these different elements via one (or more) communication bus(es) allowing the transmission of control signals, and the receipt of acquisition data derived from the force sensor 3, of the acquisition system 4, etc.

The monitoring device 5 can be composed of one (or more) workstation(s), and/or one (or more) computer(s). The monitoring device 5 comprises a processor programmed to allow the piloting of the various elements of the therapy apparatus, and to allow the processing of the signals acquired by the force sensor 3 and the acquisition system 4.

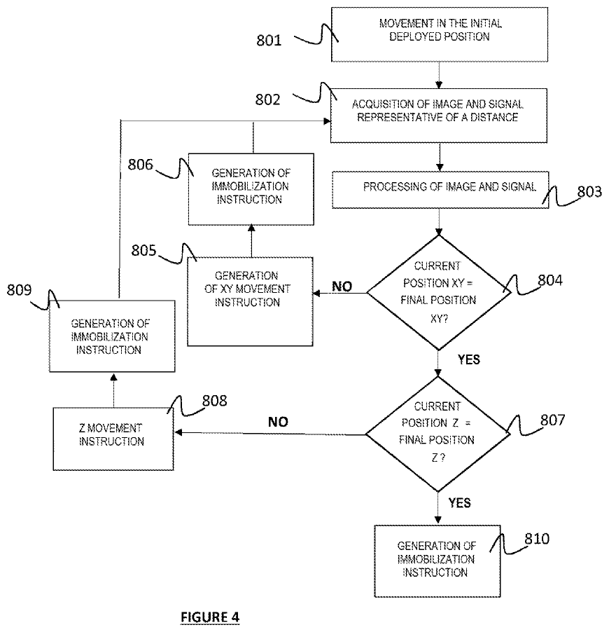

The monitoring device 5 is programmed to implement the method illustrated in FIG. 4. To this end, the monitoring device 5 comprises:
means for controlling the acquisition system 4,
means for processing each measurement pair acquired by the acquisition system 4, and
servo-control means for generating instructions to move and immobilize the arm 2.

The control means allow activating the acquisition system 4 to acquire a plurality of measurement pairs successively over time. More specifically, after each emission of an immobilization instruction by the servo-control means, the control means emit an activation signal from the acquisition system for the acquisition of a new measurement pair. This new measurement pair is processed by the processing means in order to update the deviation between the current position of the end of the arm and its desired final position.

The processing means are able, from each acquired measurement pair, to detect the three-dimensional position of the ocular tissue and the three-dimensional position of the end of the arm.

The three-dimensional position of the free end of the arm is known by construction.

The three-dimensional position of the ocular tissue is for its part obtained by calculation from the measurement pair derived from the acquisition system 4. For example in the image acquired by the acquisition system 4, the processing means are capable of identifying the ocular tissue, its two-dimensional position and its center by recognizing a shape close to a typical morphology of an eye (three concentric circles: a white circle (the sclera), in the center of which there is a colored circle (the iris) in the center of which there is a black circle (the pupil)). The third coordinate required to estimate the three-dimensional position of the ocular tissue is deduced from the signal acquired by the ranging unit, this signal being representative of the distance between the free end of the arm and the ocular tissue.

To process each measurement pair received from the acquisition system 4, the processing means comprise:
means for estimating, from the current measurement pair, the vertical distance along the axis Z between the end of the arm and the ocular tissue,
means for calculating, from the image of the current measurement pair, a horizontal deviation between:
   the current horizontal position of the free end of the arm in the horizontal plane XY, and
   the desired final horizontal position of the free end of the arm in the horizontal plane XY.

The servo-control means are programmed to implement a servo-control loop in the plane XY and a servo-control loop along the direction Z.

Advantageously, the movement of the free end of the arm 2 along the axes XY is uncorrelated from its movement along the axis Z. particularly the monitoring device 5 is programmed to:
firstly move the free end of the arm 2 in a horizontal plane XY to position said free end in the desired final horizontal position, preventing any movement of the free end along the vertical axis Z (i.e. without bringing the free end of the arm to the ocular tissue),
secondly move the free end of the arm 2 along the vertical axis Z to bring it closer to the ocular tissue until obtaining a contact, by preventing any movement of the free end in the horizontal plane XY.

This allows avoiding any risk of injury to the patient (for example by friction of the free end of the arm on the patient's eye if a movement in the plane XY was ordered while the end is already in contact with the ocular tissue).

The servo-control means of the monitoring device 5 are able to generate a plurality of successive movement instructions to move the free end of the arm from the initial deployed position to the desired final position in which the immobilization member is centered and in contact with the ocular tissue to be treated.

More specifically, if the distance between the current position of the end of the arm 2 and the desired final position is greater than a threshold value, the servo-control means generate a plurality of successive elementary movement instructions for bringing the end of the arm in the desired final position.

Between each emission of an elementary movement instruction, the servo-control means generate an immobilization instruction, and the control means emit an activation signal from the acquisition system 4 in order to acquire a new measurement pair. This allows verifying, throughout the movement of the arm 2, that its end is approaching the desired final position, and taking into account any unexpected movement of the patient's head (in which case the desired final position is updated).

### 7. OPERATING PRINCIPLE

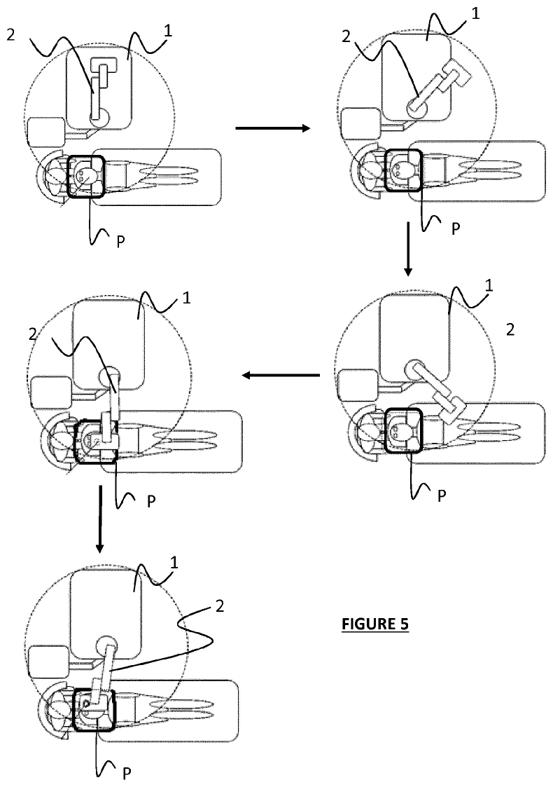

The principle of operation of the therapy equipment will now be described in more detail with reference to FIGS. 4 and 5.

7.1. Prior to the Use of the Therapy Apparatus

As a pre-condition for the proper operation of the therapy apparatus described above, it should be specified that it will be required everywhere this apparatus is used, to define the position of the surgical equipment in the room with a floor marking, which position will be defined based on:
the surgeon's preferences (right or left position, rather oriented in front, lateral or rather behind, rather close or distant)
the usual final position of the bed on which the patient is lying
the shape of the bed, its dimensions, its height
the compliance with a distance constraint, by making sure that the final position of the patient's head is within a perimeter centered around the point of attachment of the robotic arm on the surgical equipment, symbolizing its working range, or distance beyond which the arm can no longer reach its target.

Once the floor marking has been defined, the therapy apparatus will be positioned in the same location at each use. In this way, the relative position of each patient with respect to the machine and in particular of his head and his eyes, is known with an acceptable margin of error, which can go up to 20 centimeters.

Thus, it is possible, by parameterization accessible via the man-machine interface of the apparatus, to define the coordinates of a perimeter P corresponding to a square with a side of 50 cm, in which will be positioned the head of each patient preparing to receive ocular therapy with the system object of the present invention (perimeter P of certain presence of the target). Once the coordinates of this perimeter P have been stored, at each use, the monitoring device 5 controls the positioning of the end of the arm (by default and before the iterations required to obtain perfect centering) in the middle of the perimeter P. This position corresponds to the initial deployed position.

The centering and contacting the immobilization member on the ocular tissue are carried out as follows.

7.2. Automatic Positioning of the Free End of the Arm on the Ocular Tissue

7.2.1. Deployment of the Arm

Once the patient is installed and the therapy apparatus is in place, the monitoring device 5 controls the deployment of the arm 2 (step 801).

The arm 2 moves automatically (as illustrated in the first four steps of FIG. 5) so as to position the free end of the arm 2 in the center of the perimeter P (initial deployed position).

Once the center of the perimeter P has been reached, the iterations of the servo-control loop XY are initiated.

7.2.2. XY Servo-Control Loop

The XY servo-control loop is a programmed function which, at each iteration:
receives an image,
the analysis,
identifies the coordinates XY of the desired final horizontal position,
determines the coordinates X′, Y′ of the current horizontal position of the free end of the arm 2, and
calculates the deviation between the current horizontal position and the desired final horizontal position,
calculates the remaining path between the current horizontal position and the desired final horizontal position,
sends to the arm 2 one (or more) movement instruction(s) to put the arm 2 in movement along the path determined by calculation, and this until the analysis of the received image determines that the desired final horizontal position is reached (X=X′ and Y=Y′): the free end of the arm 2 is then aligned with a vertical axis passing through the center of the ocular tissue.

More specifically, the control means of the monitoring device 5 emit an activation signal from the acquisition system 4. The acquisition system 4 acquires an image and a signal representative of the distance between the end of the arm and the ocular tissue.

The processing means receive the measurement pair acquired by the acquisition system 4 and process it (step 803).

Particularly, the processing means:
detect the ocular tissue in the acquired image,
determine the position of the center of the ocular tissue,
define this position of the center of the ocular tissue as corresponding to the desired final horizontal position,
estimate the current horizontal position of the free end of the arm, and
compare (step 804) the current horizontal position with the desired final horizontal position (for example by calculating the distance between the current horizontal position and the desired final horizontal position).

The result of this comparison is transmitted to the servo-control means which:
control the implementation of the Z servo-control loop if the current horizontal position coincides with the desired final horizontal position,
generate an instruction to horizontally move the arm 2 otherwise (step 805).

Once the arm 2 has been moved in accordance with the movement instruction, the servo-control means generate an instruction to immobilize (step 806) the arm 2 and the previous steps (of activating the acquisition system 4, processing the measurement pair, etc.) are repeated until the desired final horizontal position in XY is reached by the free end of the arm 2.

7.2.3. Z Servo-Control Loop

Once the free end of arm 2 has been aligned in XY with the desired final horizontal position, the Z servo-control loop can be implemented.

The Z servo-control loop is a programmed function which, at each iteration:
receives a current altitude data from the end of the arm 2 relative to the ocular tissue (current position Z′—desired position Z),
calculates the deviation between the current vertical position of the end of the arm and the desired final vertical position,
calculates the remaining path between current vertical position and desired final vertical position,
sends to the arm 2 one (or more) movement instruction(s) to put the arm 2 in movement on the axis Z, without changing position XY, along the path determined by calculation, and this until the force sensor 3 detects a contact reflecting the fact that the desired final vertical position is reached (Z′=Z): the free end of the arm 2 is then in contact with the ocular tissue.

More specifically, the processing means of the monitoring device 5 process the signal representative of a vertical distance along the axis Z (step 803), and compare (step 807) the current vertical position with the desired final vertical position.

The result of this comparison is transmitted to the servo-control means which also receive a signal measured by the force sensor 3. The servo-control means:
generate an instruction to immobilize the arm 2 if the current vertical position coincides with the desired final vertical position (step 810),
generate an instruction to vertically move the arm 2 otherwise (step 808).

Once the arm 2 has been moved in accordance with the vertical movement instruction, the servo-control means generate an instruction to immobilize (step 809) the arm 2 and the previous steps are reiterated, including the steps of the XY servo-control loop, in order to check that the current horizontal position always corresponds to the desired final horizontal position.

This allows taking into account possible movements of the patient during the procedure for positioning the arm 2.

The monitoring device 5 allows positioning the free end of the arm in an accurate and centered manner. This free end carries the various working components allowing the treatment of the ocular tissue.

In a useful and reassuring way for the practitioner, the sequence of the different steps illustrated in FIG. 4 can be monitored by a control pedal, and/or by a voice command and/or by a tactile or non-tactile man-machine interface.

### 8. CONCLUSIONS

The invention described above allows, in a few seconds, automatically positioning on the eye of a patient a member for immobilizing the eyeball, without human intervention, in a rapid, accurate and repeatable manner. Its performances are independent of the environment, in order to gain accuracy, to make the gesture reproducible regardless of the patient or of the operator and to save time by dispensing the operator from a low-value-added task.

The invention further allows providing more safety and therefore reducing the risk run by the patient at the time of the intervention.

The reader will understand that many modifications can be made to the invention described above without physically departing from the new teachings and advantages described here. For example, in the description above, the immobilization member was mounted on the free end of the robotic arm. Alternatively, the immobilization member can be separated from the robotic arm. In this case, the immobilization member is positioned on the patient's eye prior to the movement of the robotic arm, and the desired final position corresponds to contacting the free end of the robotic arm with one face of the immobilization member opposite to the surface of the immobilization member in contact with the eye. Consequently, all modifications of this type are intended to be incorporated within the scope of the appended claims.

1. A monitoring device for monitoring the movement of an ocular therapy apparatus of the type comprising:
a support arm including a free end intended to come in line with an ocular tissue, said arm being articulated to allow the movement of the free end of the arm along three axes X, Y and Z which are orthogonal two by two:
wherein the X axis defines a longitudinal direction extending horizontally,
wherein the Y axis defines a transverse direction extending horizontally, the X axis and the Y axis defining a horizontal plane XY,
and wherein the Z axis defines a vertical direction, perpendicular to the horizontal plane XY,
an acquisition system mounted on the arm for the acquisition of a measurement pair including:
an image of the ocular tissue, and
a signal representative of a vertical distance along the Z axis between the end of the arm and the ocular tissue,
wherein the monitoring device comprises:
a controller which controls the acquisition system to acquire a plurality of measurement pairs successively over time,
a processor which processes each measurement pair, said processor including:
an estimator which estimates, from the current measurement pair, the vertical distance along the Z axis between the end of the arm and the ocular tissue,
a calculator which calculates, from the image of the current measurement pair, a horizontal deviation between:
a current horizontal position of the free end of the arm in the horizontal plane XY, and
a desired final horizontal position of the free end of the arm in the horizontal plane XY,
a servo-controller:
which generates, if the calculated horizontal deviation is greater than a first threshold value, an instruction to horizontally move the arm in the horizontal plane XY in order to reduce the deviation between the current horizontal position and the desired final horizontal position,
which generates, if the calculated horizontal deviation is less than the first threshold value and if the estimated vertical distance is greater than a second threshold value, an instruction to vertically move the arm (2) along a vertical direction in order to reduce the distance between the free end of the arm and the ocular tissue,
which generates, if the calculated horizontal deviation is less than the first threshold value and if the measured vertical distance is less than the second threshold value, an instruction to immobilize the arm.

2. The monitoring device according to claim 1, wherein the calculator comprises:
a detector which detects, from the image of the current measurement pair, the horizontal position of at least one point of interest of the ocular tissue,
an evaluator which evaluates, from the detected horizontal position of the point of interest, a horizontal deviation between:
the current horizontal position of the free end of the arm in the horizontal plane XY, and
the desired final horizontal position of the free end of the arm in the horizontal plane XY.

3. The monitoring device according to claim 2, wherein the detector identifies the ocular tissue in the acquired image, by the implementation of a shape recognition algorithm in order to detect three concentric circles in the image.

4. The monitoring device according to claim 1, wherein the ocular therapy apparatus further comprises a force sensor mounted on the free end of the arm to measure a mechanical force applied to the free end of the arm, wherein:
the processor comprises a comparator which compares said measured mechanical force with a third threshold value to determine whether the free end of the arm is in contact with an element that obstructs a vertical movement of the arm along the Z axis,
the servo-controller generates an instruction to immobilize the arm if the measured mechanical force is greater than the third threshold value.

5. The monitoring device according to claim 1, wherein the acquisition system comprises, for the acquisition of a signal representative of a vertical distance along the Z axis:
means for acquiring by laser ranging, and/or
means for acquiring by ultrasounds
means for acquiring by image processing.

6. The monitoring device according to claim 1, wherein the servo-controller generates elementary movement instructions to allow the movement of the arm between its current position and the desired final position, wherein said servo-controller generates an immobilization instruction subsequent to each elementary movement instruction.

7. A monitoring method for monitoring the movement of an ocular therapy apparatus of the type comprising:
a support arm including a free end intended to come in line with an ocular tissue, said arm being articulated to allow the movement of the free end of the arm along three axes X, Y and Z which are orthogonal two by two:
wherein the X axis defines a longitudinal direction extending horizontally,
wherein the Y axis defines a transverse direction extending horizontally, the X axis and the Y axis defining a horizontal plane XY,
and wherein the Z axis defines a vertical direction, perpendicular to the horizontal plane XY,
an acquisition system mounted on the arm for the acquisition of a measurement pair including:
an image of the ocular tissue, and
a signal representative of a vertical distance along the Z axis between the end of the arm and the ocular tissue,
wherein the monitoring method comprises the following phases:
acquiring a plurality of measurement pairs successively over time via the acquisition system,
processing each measurement pair, the processing phase comprising the steps consisting of:
estimating, from the current measurement pair, the vertical distance along the Z axis between the end of the arm and the ocular tissue,
calculating, from the image of the current measurement pair, a horizontal deviation between:
a current horizontal position of the free end of the arm in the horizontal plane XY, and
a desired final horizontal position of the free end of the arm in the horizontal plane XY,
servo-controlling the movement of the arm by:
generating, if the calculated horizontal deviation is greater than a first threshold value, an instruction to horizontally move the arm in the horizontal plane XY in order to reduce the deviation between the current horizontal position and the desired final horizontal position,
generating, if the calculated horizontal deviation is less than the first threshold value and if the estimated vertical distance is greater than a second threshold value, an instruction to vertically move the arm along a vertical direction in order to reduce the distance between the free end of the arm and the ocular tissue,
generating, if the calculated horizontal deviation is less than the first threshold value and if the measured vertical distance is less than the second threshold value, an instruction to immobilize the arm.

8. The monitoring method according to claim 7, wherein the calculation step includes the following sub-steps:
detecting, from the image of the current measurement pair, the horizontal position of at least one point of interest of the ocular tissue,
evaluating, from the detected horizontal position of the point of interest, a horizontal deviation between:
the current horizontal position of the free end of the arm in the horizontal plane XY, and
the desired final horizontal position of the free end of the arm in the horizontal plane XY.

9. The monitoring method according to claim 8, wherein the detection sub-step consists in identifying the ocular tissue in the acquired image, by the implementation of a shape recognition algorithm to detect three concentric circles in the image.

10. The monitoring method according to claim 7, wherein the therapy apparatus further comprises a force sensor mounted on the free end of the arm for measuring a mechanical force applied to the free end of the arm:
the processing phase comprising a step of comparing said measured mechanical force with a third threshold value to determine whether the free end of the arm is in contact with an element that obstructs a vertical movement of the arm along the Z axis,
the servo-control step including the generation of an instruction to immobilize the arm if the measured mechanical force is greater than the third threshold value.

11. The monitoring method according to claim 7, wherein the acquisition phase comprises:
the acquisition, by laser ranging, of a signal representative of a vertical distance along the Z axis, and/or
the acquisition, by ultrasounds, of a signal representative of a vertical distance along the Z axis, and/or,
the extraction of an acquired image from a signal representative of a vertical distance along the Z axis.

12. The monitoring method according to claim 7, wherein the servo-control step includes:
generating an elementary movement instruction to allow the movement of the arm between its current position and a desired final position,
generating an immobilization instruction subsequent to each elementary movement instruction,
repeating the previous sub-steps until the calculated horizontal deviation is less than the first threshold value and the measured vertical distance is less than the second threshold value.
